(19) United States
(12) Patent Application Publication
(10) Pub. No.: US 2014/0361774 A1
(43) Pub. Date: Dec. 11, 2014

(54) METHOD FOR QUANTITATIVE DETERMINATION OF SODIUM IN PETROLEUM FUEL

(71) Applicant: Nanonord A/S, Alborg (DK)

(72) Inventor: Ole Norgaard Jensen, Alborg (DK)

(21) Appl. No.: 14/364,361

(22) PCT Filed: Dec. 7, 2012
(86) PCT No.: PCT/DK2012/050452

§ 371 Date: Jun. 11, 2014

(30) Foreign Application Priority Data
Dec. 12, 2011 (DK) PA 2011 00964
Sep. 4, 2012 (DK) PA 2012 70535

(51) Int. Cl. G01N 24/08; G01N 33/22; G01R 33/58; G01R 33/46; G01R 33/48; G01R 33/54

(52) U.S. Cl. CPC G01N 24/08; G01R 33/4828; G01R 33/543; G01R 33/58; G01R 33/4616; G01N 33/22
USPC 324/309; 324/318

(57) ABSTRACT

The invention relates to a method of and a system for quantitative determination of sodium in petroleum fuel, such as heave fuel oil. The method comprises determining a concentration of sodium in the petroleum fuel using NMR. The method advantageously comprises determining sodium in the form of sodium isotope ²³Na by performing at least one NMR measurement on at least a part of the petroleum fuel, obtaining at least one NMR spectrum from the NMR measurement(s) and performing the quantitative determination of sodium based on the NMR spectrum, where the result is compared to calibration data comprising NMR—determinations on petroleum fuels with known sodium concentration.

### TECHNICAL FIELD

The invention relates to a method and a system for quantitative determination of sodium in petroleum fuel, in particular heavy fuel oil or other types of inhomogeneous petroleum fuels.

### BACKGROUND ART

Petroleum fuel often comprises traces of contaminants, such as sodium, potassium, calcium, lead and vanadium. Depending on the use of the petroleum fuel it may be required to ensure that the content of one or more of such contaminants are limited below a very low limit. Several of these contaminants are highly corrosive if the petroleum fuel is used in combustion engines, such as gas turbines where the temperature becomes very high.

It is well known to treat the petroleum fuel to remove or inhibit more or less of the corrosive contaminant. A method for removal or inhibiting is for example described in CA 1085614. Other methods include addition of magnesium to inhibit vanadium. Alkali contaminants are normally removed by washing process' which includes addition of water and subsequent removal of water with dissolved contaminants e.g. by centrifugation. This may often require several washing process to bring the amount of in particular sodium down to an acceptable level, which at present is 1 ppm or less.

The amount of sodium in the petroleum fuel can be determined by analyzing samples in laboratories. Since the petroleum fuel is often very inhomogeneous it is required to take several samples to obtain an at least fairly reliable result.

Since it is both burdensome and time demanding to take out sample, bring them to the laboratory for test and performing the test, methods and instruments for testing for sodium and other contaminant on the site by flame spectrometry has been developed. U.S. Pat. No. 6,268,913 describe such system where a flame spectrometer senses the level, such as the concentration level, of a fuel contaminant, such as sodium, within the combustion flame and a control system disables the fuel delivery system as a function of the contaminant's concentration level or accumulated concentration level. This method is however not sufficient, since it is very burdensome to stop the combustion and it is generally desired to avoid introducing petroleum fuel with too high level of sodium into the turbine.

ASTM D5863 discloses a standard method for determining the concentrations of vanadium, nickel, iron, and sodium in crude oils and residual fuels by flame atomic absorption spectrometry on samples.

The object of the invention is to provide a new and reliable method and system of quantitative determination of sodium in petroleum fuel, which method is simultaneously fast and is suitable for on-site determinations, such that it is not required to bring samples to a laboratory.

### DISCLOSURE OF INVENTION

This object has been solved by the present invention as defined in the claims. The method of the invention for quantitative determination of sodium in petroleum fuel and embodiments thereof as well as the system of the invention for quantitative determination of sodium in petroleum fuel has shown to have a large number of advantages which will be clear from the following description.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

As explained above, before the present invention was conceived, quantitative determination of sodium in petroleum fuel has been very difficult and cumbersome and/or generally required a destructive test i.e. the petroleum fuel or samples thereof needed to be combusted in order to test for sufficiently low amounts of sodium e.g. in the order of ppm, such as less than 1 ppm. The present invention provides a highly improved and non-destructive method which is very reliable and fast, can measure on large amounts of the petroleum fuel e.g. all of the petroleum fuel instead of just samples, and which simultaneously is suitable for on-site determinations e.g. before or after washing of the petroleum fuel, prior to injecting into a turbine or in principle anywhere along a flow line of petroleum fuel.

The determination of sodium in a petroleum fuel can advantageously be performed on board a vessel such as it will be described further below. In practice the method of the invention has shown to be operable in nearly real-time.

In an embodiment of the invention the method is applied to provide a level of the sodium in a petroleum fuel also referred to as a fuel oil.

In an embodiment of the invention the method is applied to determine the total content or concentration of sodium in a petroleum fuel.

It has been found that by use of the method of the invention, even very small amounts, such as less than 1 ppm sodium bound in compound(s) and/or on ionic form can be determined with a very high accuracy.

Even though the phenomenon of Nuclear Magnetic Resonance (NMR) is well known and is also well known to apply in determination of isotopes by spectroscopy e.g. for use in determining organic compounds using proton ¹H NMR or ¹³C NMR, it has heretofore never been suggested or even considered possible to apply NMR in determination of sodium in such small amounts in petroleum fuel.

In an embodiment the method of the invention is for use in a laboratory, which provides an alternative and cost effective and non-destructive method compared to prior art methods.

The method of performing a quantitative determination of sodium in petroleum fuel using NMR has shown to be very fast and reliable and it can thereby be avoided to introduce petroleum fuel with undesired high level of sodium into a combustion chamber, such as a into a gas turbine engine without undesired delay.

As it will be explained in more detail below, the signal obtained in the sodium determination can be correlated directly to the amount of sodium in the petroleum fuel measured on, and since the petroleum fuel often is rather inhomogeneous it is desired to measure on a large amount of the petroleum fuel e.g. all of the petroleum fuel, which accordingly can be performed in a simple and non-destructive way.

The method of the invention is in particular advantageous where the petroleum fuel is inhomogeneous e.g. fuel that is or comprises a heavy fuel oil (HFO), such as bunker fuel oil.

Nuclear magnetic resonance—abbreviated NMR—is a phenomenon which occurs when the nuclei of certain atoms are immersed in a static magnetic field and exposed to a second oscillating magnetic field. NMR measurement is performed by NMR spectroscopy and comprises using the NMR phenomenon to study materials e.g. for analyzing organic chemical structures

The method of the invention preferably comprises determining sodium in the form of sodium isotope ²³Na by performing at least one NMR measurement (also referred to as NMR spectroscopy) on at least a part of the petroleum fuel, obtaining at least one NMR spectrum from the NMR measurement(s) and performing the quantitative determination of sodium based on the NMR spectrum. The term “NMR spectrum” is herein used to designate the signal obtained from an NMR measurement. The NMR spectrum may be in the form of part(s) of or all of a physical drawn spectrum, it may be in the form of part(s) of or all of a spectrum in digital form, it may be in the form of peak determinations or results derived there from or in any other form in which the resulting signals or parts thereof obtained from an NMR measurement can be provided. Such NMR spectra are well known in the art.

In an embodiment of the method the one or more NMR spectra obtained are used to perform at least one quantitative sodium determination. The at least one quantitative sodium determination can for example be a determination of a sodium concentration and/or amount in a fraction of a petroleum fuel, a determination of a sodium concentration and/or amount in a batch of a petroleum fuel, a determination of a concentration and/or amount of sodium ion or a specific sodium containing compound, a determination of a level of sodium i.e. if it is above or below a selected threshold, such as a threshold of about 1 ppm.

Spectrometers are well known in the art and the skilled person will be able to select a suitable spectrometer for use in the present invention based on the teaching provided herein. Examples of spectrometer are e.g. described in U.S. Pat. No. 6,310,480 and in U.S. Pat. No. 5,023,551.

A spectrometer comprises a unit for providing a permanent field e.g. a permanent magnet assembly as well as a transmitter and a receiver for transmitting and/or receiving RF frequency pulses/signals The RF receiver and RF transmitter are connected to an antenna or an array of RF antennae, which may be in the form of transceivers capable of both transmitting and receiving. The spectrometer further comprises at least one computing element, in the following referred to as a computer.

General background of NMR formation evaluation can be found, for example in U.S. Pat. No. 5,023,551.

Although ‘NMR measurement’ in the following often will be used in singular to describe the invention, it should be observed that the singular term ‘NMR measurement’ also includes a plurality of NMR measurements unless other is specified.

In an embodiment of the invention the NMR measurement is performed on the petroleum fuel in flowing condition. The NMR measurement may for example be performed on the petroleum fuel during transportation from a first container to a second container or to a point of use, such as to a second storage container or to use in an engine e.g. a turbine.

The second storage container can for example be a storage tank, a refinery or a service tank. In an embodiment the NMR measurement is performed on the petroleum fuel in flowing condition in a pipe section pumping the fuel from a first container and back to the same first container. In this embodiment the method can advantageously comprising sending high sodium content petroleum fuel fractions to a washing step for reducing the sodium concentration and thereafter returning the washed petroleum fuel to the first container.

In an embodiment the NMR measurement is performed on the petroleum fuel in flowing condition in a pipe section pumping the petroleum fuel from a first container to a second container e.g. of a refinery or a second storage container or to a point of use, such as to use in a turbine.

When performing the NMR measurement on the petroleum fuel in flowing condition it should advantageously be ensured that the velocity of the flowing fuel is adjusted or kept such that the fuel part is within the spectrometer range for a sufficient time to perform the NMR measurement.

In an embodiment of the invention the NMR measurement is performed in-line or semi-in-line, comprising performing the NMR measurement on-site, such as on-site of a gas turbine or on-site of washing equipment for washing off sodium. The NMR measurement may advantageously be performed onboard a motor driven unit, such as a vessel.

In an embodiment the NMR measurement is performed in-line directly on the petroleum fuel in flowing condition e.g. in a pipe or directly on the petroleum fuel in a container comprising the petroleum fuel.

The term “in-line” should herein be interpreted to mean that the NMR measurement is performed directly on the petroleum fuel without removing the petroleum fuel part (i.e. a sample of the petroleum fuel) from the remaining petroleum fuel. The NMR measurement may e.g. be performed on the petroleum fuel in flowing condition as described above or it may be performed directly on the petroleum fuel in a container e.g. near the bottom of a container comprising the petroleum fuel since the sodium concentration often will be higher near the bottom of a container, than near the surface of the petroleum fuel in a container if the petroleum fuel is not subjected to tubules such as stirring.

The term “semi-in-line” should herein be interpreted to mean that NMR measurement is performed on the petroleum fuel sample temporally withdrawn from the remaining fuel, performing at least one NMR measurement and optionally returning the petroleum fuel sample to the remaining part of the petroleum fuel. A plurality of consecutive NMR measurements on consecutively withdrawn fuel samples are performed, such that a representative amount of the petroleum fuel is subjected to determination. Preferably at least some of the fuel samples, such as about 90% or more of the fuel parts are returned to the remaining petroleum fuel. In case a sample has very high sodium concentration it may be advantageous to discharge this sample instead of returning it to the remaining petroleum fuel.

In an embodiment the NMR measurement is performed semi-in-line by temporally withdrawing petroleum a fuel part as a sample of the petroleum fuel, performing the NMR measurement on the withdrawn sample and optionally returning the sample to the remaining petroleum fuel. The petroleum fuel sample may advantageously be withdrawn from a container comprising the petroleum fuel onboard the motor driven unit.

According to the invention it has been found to be very beneficial that the NMR measurement can be performed directly onboard a vessel. The determination of sodium can be performed as a continuous process which thereby provides a very economically attractive system, which simultaneously provides a high safety against sodium destroying parts of the engines or other equipment in contact with the petroleum fuel due to corrosion.

In an embodiment the NMR measurement comprises withdrawing a petroleum fuel part as a sample of the petroleum fuel and performing the NMR measurement on the withdrawn sample. In this embodiment the petroleum fuel sample may e.g. be sent to a laboratory for having the NMR measurement performed.

In an embodiment of the invention comprising onboard determination of sodium is combined with ad hoc controlling laboratory sodium determinations which laboratory sodium determinations may be performed using traditional prior art methods such ad flame spectroscopy or which laboratory sodium determinations may be performed using NMR according to the invention. For cost effective determinations the latter method will generally be preferred.

In an embodiment of the invention the method comprises repetitive determinations of sodium, e.g. for observing changes of type, level and/or level of sodium component(s)/sodium ions in the petroleum fuel.

In an embodiment the method comprises determining sodium in the petroleum fuel, subjecting the fuel to a sodium removal treatment, and repeating the determination of sodium in the petroleum fuel.

The sodium removal treatment can be any kind of extracting or cleaning treatment suitable for extracting sodium from petroleum fuel, such as heavy fuel oil or diesel. The extraction treatment may for example be performed by washing as described above, i.e. by adding water, allowing sodium to be dissolved/dispersed in the water e.g. by thorough mixing of petroleum fuel and water, and removing the water e.g. using suitable centrifuges and/or using a settling tank or by using other types of separators.

The sodium removal treatment is advantageously provided to reduce the amount of sodium to below 1 ppm. Optionally the sodium removal treatment is repeated to reach below this threshold. This method will be described in further detail below.

In an embodiment NMR measurement comprises simultaneously subjecting the petroleum fuel to a magnetic field B and a plurality of pulses of radio frequency energy E (in form of RF pulses) and receiving electromagnetic signals from the ²³Na isotope isotopes. The method preferably comprises determining the concentration of ²³Na isotope in the petroleum fuel measured on e.g. a petroleum fuel sample.

RF pulses mean herein pulses of radio frequency energy.

In order to obtain NMR spectra of a high resolution (i.e. as low noise as possible) it is generally desired that the NMR measurements are performed using a relatively high magnetic field B.

In an embodiment the magnetic field B is at least about 1 Tesla, such as at least about 1.2 Tesla, such as at least about 1.4 Tesla, such as at least about 1.6 Tesla.

The magnetic field B may be generated by any suitable means. The magnetic field B is in a preferred embodiment between about 1 and about 3 Tesla, such as between about 1.5 and 2.5 Tesla.

In an embodiment the magnetic field is generated by a permanent magnet, such as a neodymium magnet. Since permanent magnets are generally not costly, this solution provides a low cost solution which for many applications may provide a sufficient low noise result.

In an embodiment the magnetic field is generated by an electromagnet, such as a solenoid magnet or other electromagnets which are usually applied in motors, generators, transformers, loudspeakers or similar equipment. Electromagnets of high strength e.g. electromagnets that can be applied for generating a field of about 1.5 Tesla or more are often relatively expensive compared with permanent magnets. However, the magnetic field generated using electromagnets can be both relatively strong and relatively homogeneous simultaneously, which is very beneficial in the present invention.

Furthermore, the electromagnet may be adjusted by adjusting the current in the coil of the electromagnet to a desired level.

In a preferred embodiment the magnetic field is generated by an electromagnet in form of a superconducting magnet comprising a coil of superconducting wire. Such superconducting magnets are well known in the art and can be made to produce relatively high magnetic fields. Furthermore such superconducting magnets can provide a very homogeneous field and simultaneously they are relatively cheaper to operate because almost no energy dissipates as heat in windings of the coils.

Examples of superconducting magnets suitable in the present invention are disclosed in GB 2474343 or in GB 2467527.

In an embodiment of the invention the magnetic field in the measuring zone, i.e. the part where the petroleum fuel part to be measured on is located when the NMR measurement is performed, is preferably relatively spatially homogeneous and relatively temporally constant. However, in general it is difficult to provide that the magnetic field in the measuring zone is entirely homogenous and further for most magnetic fields, the field strength might drift or vary over time due to aging of the magnet, movement of metal objects near the magnet, and temperature fluctuations.

Drift and variations over time can be dealt with by controlling temperature and/or by applying a field lock such as it is generally known in the art.

Spatial inhomogeneities of the magnetic field can be corrected for by a simple calibration or alternatively or simultaneously such spatial inhomogeneities can be adjusted for by shim coils such as it is also known in the art. Such shim coils may e.g. be adjusted by the computer to maximize the homogeneity of the magnetic field.

In an embodiment of the invention the method comprises performing a plurality of NMR measurement at a selected magnetic field, preferably the magnetic field is kept substantially stationary during the plurality of NMR measurements.

In an embodiment the method of the invention comprises regulating the temperature e.g. by maintaining the temperature at a selected value.

In an embodiment the method of the invention comprises determining the temperature.

The term ‘substantially’ is herein used to include ordinary variations and tolerances which are normally accepted within the art in question.

In an embodiment the method comprises performing a plurality of NMR measurement on the petroleum fuel. Advantageously the NMR measurement of the petroleum fuel will be performed a plurality of times in order to reduce the noise. In an embodiment the NMR measurement is performed continuously in repeated measuring cycles. In an embodiment the method comprises performing a plurality of NMR measurements on the same petroleum fuel part (e.g. a sample). The NMR measurements are normally performed very fast e.g. several NMR measurement circles per second, such as 20 NMR measurements or more, such as 50 NMR measurements or more. Therefore even when performing the NMR measurement on the petroleum fuel part in flowing condition, several NMR measurements may be performed on virtually the same petroleum fuel part.

In an embodiment the NMR measurement comprises simultaneously subjecting the petroleum fuel part to a magnetic field B, and an exciting RF pulse with frequencies selected to excite a nuclei spin of at least a part of the ²³Na isotope. Preferably the exciting RF pulse has a band width (span over a frequency range) which is sufficient to excite at least one nuclei spin (spin transition) of substantially all ²³Na isotopes in the petroleum fuel part.

In theory one single exciting RF pulse can be sufficient to obtain a useful signal. In an embodiment of the invention it is desired to use a sequence of RF pulses with frequencies having a selected band width in order to excite a desired number of nuclei spin of the sodium isotopes in the petroleum fuel part.

A general background description of NMR measurement can be found in “NMR Logging Principles and Applications” by George R. Coates et al, Halliburton Energy Services, 1999. See in particular chapter 4. Although this document does not specifically describe the NMR determination of sodium isotope, the principle applied is similar.

Sodium comprises twenty recognized isotopes, ranging from ¹⁸Na to ³⁷Na. According to the invention it has been found that NMR determinations based on the ²³Na isotope provides the most reliable determination.

The ²³Na isotope has an electric quardrupole moment of about 10.4×10⁻³⁰ m². It has several nuclei spins which may be excited at equal or at different frequencies in dependence on the environment of the ²³Na isotope, i.e. the compound it is part of or as ion. It is said that the nuclei spins of the ²³Na isotope are shifted due to quadrupolar couplings when the nuclei spins of the sodium isotope are excited at different frequencies.

Quadrupole Splitting reflects the interaction between the nuclear energy levels and surrounding electric field gradient (EFG). Nuclei in states with non-spherical charge distributions, such as ²³Na isotope with angular quantum number of 3/2, produce an asymmetrical electric field which splits the nuclear energy levels. This produces a nuclear quadrupole moment. The quadrupole moment interacts anisotropically (orientation dependent) with the EFG, resulting in optional splitting up of signals from an ²³Na isotope, dependent on its position in a compound or an ion and in particular dependent on the symmetry of a compound it is part of. The splitting up of signals from an ²³Na isotope is called the quadrupole broadening.

In an embodiment the petroleum fuel is subjected to an exciting RF pulse with frequencies selected to excite at least one nuclei spin of substantially all ²³Na isotopes the petroleum fuel part under determination. Preferably the petroleum fuel part is subjected to an exciting RF pulse with frequencies selected to excite the ²³Na isotopes in their central band, such that at least a central (seen in relation to the exciting frequency) nuclei spin of the ²³Na isotopes in the petroleum fuel part is excited. Nuclei spins of the sodium isotopes that are not in the central band are said to be in side bands.

In an embodiment the petroleum fuel part is subjected to an exciting RF pulse with frequencies selected to excite a plurality nuclei spins of substantially all ²³Na isotopes in the petroleum fuel part. Preferably the petroleum fuel part is subjected to an exciting RF pulse with frequencies selected to excite the ²³Na isotopes at least in their central band and at least to excite one or more nuclei spins of the ²³Na isotopes in their side bands.

In an embodiment the petroleum fuel part is subjected to an exciting RF pulse with frequencies selected to excite substantially all nuclei spins of substantially all ²³Na isotopes in the petroleum fuel part.

A sufficient frequency range of radio pulses (band width) can be found by performing a calibration test on petroleum fuels with known content of sodium that is desired to be determined. The ²³Na isotope in petroleum fuels will normally be excited at least in their central bands within a relatively small frequency range of radio pulses.

Chemical shift is defined as the relative difference in resonant frequency compared to a reference signal. The shift is believed to be caused by spin-spin coupling between protons of compounds.

Chemical shifts of the exciting due to the bonding of the ²³Na isotopes in the petroleum fuel are generally so small that such chemical shifts can be ignored.

Inhomogeneities of the magnetic field should normally also be accounted for when selecting the band width.

In an embodiment the radio frequency pulses are in form of adiabatic RF pulses, i.e. RF pulses that are amplitude and frequency modulated pulses.

As mentioned above ²³Na isotope is a spin 3/2 nucleus and is therefore quadrupolar. As a result, the signal width increases with asymmetry of the environment with small or somewhat broad lines in symmetrical environments but very broad lines in asymmetric ones. This effect is generally known in the art.

Also the presence of other components, such as water in the petroleum fuel can result in antenna detuning and provisions shall be made to automatically keep track of this tuning and adjust match, if necessary.

In an embodiment of the invention the frequency range of the exciting RF pulse spans over at least about 10000 ppm, preferably at least about 50000 ppm, such as from about 2000 ppm to about 50000 ppm.

The span of frequencies as well as a frequency shift is often measured in ppm—i.e. with respect to a reference compound.

Based on the teaching provided herein, the skilled person will be able to select a frequency range of the exciting RF pulse which is sufficient to obtain a reliable determination of sodium in a petroleum fuel.

In an embodiment of the invention the frequency range of the exciting RF pulse comprises a band width of at least about 1 MHZ.

By a few trial and error tests the desired frequency range for at specific type of determination can be found.

The actual frequencies that are exiting the spin of the ²³Na isotope nucleus depend largely on the magnetic field B. As explained above, the magnetic field may vary due to drift and due to temperature variations and it is generally preferred that the exciting RF pulses are adjusted by a field lock function in order to ensure that the NMR measurements are performed using exciting RF pulses which are directed towards desired nucleus spin of the ²³Na isotope.

For example in an embodiment where the magnetic field is from about 1 T to about 2 T, the exciting RF pulse preferably comprises at least some of the frequencies in the range from about 10 MHz to about 22 MHz, such as at least a frequency band width of at least about 1 MHZ. In an embodiment where the magnetic field is from about 1 T to about 2 T, the exciting RF pulse comprises at least some of the frequencies in the range from about 13 MHz to about 19 MHz.

In an embodiment the method of the invention comprises determining at least one relaxation rate of an exited ²³Na isotope.

The term relaxation describes processes by which nuclear magnetization excited to a non-equilibrium state return to the equilibrium distribution. In other words, relaxation describes how fast spins “forget” the direction in which they are oriented. Methods of measuring relaxation times T1 and T2 are well known in the art.

In an embodiment the method comprises determining at least one spin-lattice—T1 relaxation value of an exited ²³Na isotope.

It is believed that T1 relaxation involves redistributing the populations of nuclear spin states in order to reach the thermal equilibrium distribution.

T1 relaxation values may be dependent on the NMR frequency applied for exciting the ²³Na isotope. This should preferably be accounted for when analyzing and calibrating the T1 relaxation values obtained.

In an embodiment the method comprises determining at least one spin-spin—T2 relaxation value of an exited ²³Na isotope.

The T2 relaxation is also called the transverse relaxation.

Generally T2 relaxation is a complex phenomenon and involves decoherence of transverse nuclear spin magnetization. T2 relaxation values are substantially not dependent on the magnetic field applied during excitation of the sodium isotope, and for most determinations such possible variations can be ignored.

In an embodiment the method comprises subjecting the petroleum fuel part to pulsed trains of RF pulses, preferably with repetition rates of at about 100 ms or less, such as from about 10 to about 50 ms, such as from about 15 to about 20 ms.

The trains of RF pulses are often applied to determine the T1 and/or T2 values.

In an embodiment, the method comprises subjecting the petroleum fuel part to trains of square RF pulses, preferably with repetition rates of about 100 ms or less, such as about 10 ms or less, such as about 5 ms or less, such as about 1 ms or less.

A short square pulse of a given “carrier” frequency “contains” a range of frequencies centered about the carrier frequency, with the range of excitation (bandwidth/frequency spectrum) being inversely proportional to the pulse duration.

In the present invention it is in an embodiment desired that the carrier frequency is from about 13 MHz to about 19 MHz and the duration is from about 5 μs to about 20 μm when the magnetic field is from about 1 to about 2 T. The frequencies can be regulated accordingly if another magnetic field is applied.

A Fourier transform of an approximately square wave contains contributions from all the frequencies in the neighborhood of the principal frequency. The restricted range of the NMR frequencies made it relatively easy to use short (millisecond to microsecond) radio frequency pulses to excite the entire NMR spectrum.

In an embodiment the NMR measurement comprises simultaneously subjecting the petroleum fuel part to a magnetic field B and a plurality of RF pulses wherein the RF pulses comprise
i. an exciting RF pulse, and
ii. at least one refocusing RF pulse.

The exciting RF pulse and the refocusing pulse or pulses may for example be in the form of a train of RF pulses, e.g. pulsed pulses. The exciting RF pulse is preferably as described above and may in an embodiment be pulsed.

Useful duration and amplitude of the exciting RF pulses are well known in the art and optimization can be done by a simple trial and error.

In an embodiment the exciting RF pulse is in the form of a 90° pulse.

A 90° pulse is an RF pulse designed to rotate the net magnetization vector 90° from its initial direction in the rotating frame of reference. If the spins are initially aligned with the static magnetic field, this pulse produces transverse magnetization and free induction decay (FID).

In an embodiment the refocusing RF pulse(s) is in the form of a 180° pulse, preferably the method comprises subjecting the petroleum fuel part to a plurality of refocusing RF pulses, such as one or more trains of refocusing RF pulses.

A 90° pulse is an RF pulse designed to rotate the net magnetization vector 180° in the rotating frame of reference. Ideally, the amplitude of a 180° pulse multiplied by its duration is twice the amplitude of a 90° pulse multiplied by its duration. Each 180° pulse in the sequence (called a CPMG sequence after Carr-Purcell-Meiboom-Gill) creates an echo.

A standard technique for measuring the spin-spin relaxation time T2 utilizing CPMG sequence is as follows. As is well known after a wait time that precedes each pulse sequence, a 90-degree exciting pulse is emitted by an RF antenna, which causes the spins to start processing in the transverse plane. After a delay, an initial 180-degree pulse is emitted by the RF antenna. The initial 180-degree pulse causes the spins, which are dephasing in the transverse plane, to reverse direction and to refocus and subsequently cause an initial spin echo to appear. A second 180-degree refocusing pulse can be emitted by the RF antenna, which subsequently causes a second spin echo to appear. Thereafter, the RF antenna emits a series of 180-degree pulses separated by a short time delay. This series of 180-degree pulses repeatedly reverse the spins, causing a series of “spin echoes” to appear. The train of spin echoes is measured and processed to determine the spin-spin relaxation time T2.

In an embodiment the refocusing RF pulse(s) is/are applied with an echo-delay time after the exciting RF pulse. The echo-delay time (also called wait time TW) is preferably of about 500 μs or less, more preferably about 150 μs or less, such as in the range from about 50 μs to about 100 μs.

This method is generally called the “spin echo” method and was first described by Erwin Hahn in 1950. Further information can be found in Hahn, E. L. (1950). “Spin echoes”. Physical Review 80: 580-594, which is hereby incorporated by reference.

A typical echo-delay time is from about 10 μs to about 50 ms, preferably from about 50 μs to about 200 μs. The echo-delay time (also called wait time TW) is the time between the last CPMG 180° pulse and the first CPMG pulse of the next experiment at the same frequency. This time is the time during which magnetic polarization or T1 recovery takes place. It is also known as polarization time.

This basic spin echo method provides very good result for obtaining T1 relaxation values by varying TW and T2 relaxation values can also be obtained by using plurality of refocusing pulses.

In an embodiment the at least one refocusing pulse comprises a plurality of refocusing pulses or trains of refocusing pulses applied with refocusing delay (TE) intervals between two consecutive refocusing pulses.

The refocusing delay is also called the Echo Spacing and indicates the time identical to the time between adjacent echoes. In a CPMG sequence, the TE is also the time between 180° pulses.

This method is an improvement of the spin echo method by Hahn. This method was provided by Carr and Purcell and provides an improved determination of the T2 relaxation values.

Further information about the Carr and Purcell method can be found in Carr, H. Y.; Purcell, E. M. (1954). “Effects of Diffusion on Free Precession in Nuclear Magnetic Resonance Experiments”. Physical Review 94: 630-638, which is hereby incorporated by reference.

A typical refocusing delay interval is from about 50 μs to about 0.1 ms, preferably about 75 μs.

In an embodiment the NMR measurement comprises a repeating exciting-refocusing sequence each exciting-refocusing sequence comprises
i. an exciting RF pulse, and
ii. at least one refocusing RF pulse.

The exciting-refocusing sequence is preferably repeated a plurality of times such as at least 100 times, such as at last 200 or preferably much more.

In order to reduce noise it is generally desired to repeat the exciting-refocusing sequence 5.000 times or more. In an embodiment of the invention the exciting-refocusing sequence is repeated with 5 to 500 exciting-refocusing sequences per second, such as with 50 to 400 exciting-refocusing sequences per second, such as with 150 to 250 exciting-refocusing sequences per second.

In an embodiment the exciting-refocusing sequence is repeated from about 5 minutes to about 24 hours, such as typically from about 1 hour to about 10 hours.

The higher magnetic field strength in the measuring zone the better the signal to noise ratio will be and in general the fewer repetitive NMR measurements are needed. In general the noise will be reduced with the square number of repeated NMR measurements.

The number of repeating NMR measurement for a given determination versus the time requires can be optimized by the skilled person.

In an embodiment of the invention the method comprises obtaining at least one NMR spectrum comprising an NMR spectrum from −500 ppm or less to +500 ppm or more, such as from −2000 ppm or less to +2000 ppm or more in relation to a reference Na-composition. The reference Na-composition is preferably a homogeneous mixture of hydrocarbon(s), water and fully dissolved sodium chloride in a known concentration, such as a homogeneous mixture of a sodium free petroleum, such as a petroleum jelly e.g. Vaseline and a 5% w/w aqueous solution of sodium chloride.

In an embodiment the method comprises determining quantitatively and/or qualitatively at least one compound comprising sodium and/or sodium ions. According to the invention it is anticipated that by comparing the result obtained by the NMR measurement of a given petroleum fuel part with corresponding NMR measurements of petroleum fuel with known sodium compounds and/or sodium ion, it can be deduced if the detected ²³Na isotope is an ion or the sodium is a part of a compound ad if so, optionally which compound.

In this connection the NMR measurements of petroleum fuel with known amounts of sodium ions and sodium containing compounds are used as a calibration map which can be stored in a computer for calibrating the NMR measurements of a given sample.

In most petroleum fuels all present sodium is in dissolved form. In heavy fuel oils such as crude oil all sodium will normally be in form of dissolved sodium chloride, and the sodium chloride will mainly be dissolved in water in the petroleum fuel. The determinations of sodium in such heavy fuel oils can therefore be performed by merely quantitatively determine the sodium ion in the heavy fuel oil, which determination has shown to provide highly reliable results. Where it is sufficient to quantitatively determine the sodium ion in the heavy fuel oil, NMR measurements of petroleum fuel with known amounts of sodium ions are used as a calibration map, e.g. stored in a computer for calibrating the NMR measurements of a given sample.

In an embodiment the method of the invention comprises determining the concentration of sodium ions, e.g. in a part of the petroleum fuel, such as a sample of the petroleum fuel or in a whole batch of petroleum fuel.

During the determination the temperature is advantageously maintained at a selected value or the method comprises simultaneously determining the temperature.

In an embodiment the temperature is maintained within a value range of 10 degrees or less, such as within the range 15-25° C.

In an embodiment method comprises providing a calibration map of petroleum fuels with known concentrations of sodium.

The term ‘calibrating map’ is herein used to designate a collection of NMR spectra data obtained in petroleum fuels with known amounts of sodium ions and optionally NMR spectra data obtained in petroleum fuels with known amounts sodium containing compounds. The calibration map may be in form of raw data, in form of drawings, in form of graphs, in form of formulas or any combinations thereof.

In an embodiment, the petroleum fuel used for generating the calibrating map is of a similar type as the petroleum fuel to be tested. In an embodiment the calibration map also comprises a plurality of values determined on an petroleum fuel mixed with additional water.

Generally it is well known in the art to calibrate NMR measurements based on NMR spectra obtained on known compositions.

In an embodiment the calibration map is in the form of a pre-processed data set, where the NMR spectra obtained for an petroleum fuel under analysis can be processed by the computer to provide a clear level, amount or concentration of sodium in the petroleum fuel.

In an embodiment the method comprises preparing calibration data and storing the calibration data on a digital memory. The method advantageously comprises feeding the NMR spectrum(s) or data obtained from the NMR spectrum(s) to a computer in digital communication with the digital memory and providing the computer to compare and analyze the data to perform at least one quantitative sodium determination.

The calibration map may be built up during use, for example additional data obtained by measurement on the petroleum fuel is fed to the computer and used in the calibration of the data for later determinations

The computer may for example be programmed to compute the data obtained using artificial intelligence or the calibration map may be applied to teach a neural network.

In an embodiment the method comprises performing at least one NMR measurement on a plurality of petroleum fuel parts, preferably the method comprises performing a plurality of NMR measurements and optionally other measurements, such as hydrogen measurements.

In order to improve the determination in the petroleum fuel, other compounds can additionally and preferably simultaneously be determined in the petroleum fuel.

In an embodiment the method further comprises performing a potassium determination of measurement on the petroleum fuel part using NMR, preferably by determining ³⁹K isotope by performing at least one NMR measurement on the petroleum fuel part, obtaining at least one NMR spectrum from the NMR measurement(s) and performing at least one quantitative and/or qualitative potassium determination. The NMR measurement preferably comprises obtaining at least one spin-lattice—T1 value and at least one spin-spin—T2 value of an exited potassium ³⁹K isotope.

Potassium determination using NMR can be performed in a similar manner as the method described above but by using other frequencies and optionally the strength of the magnetic field may also be adjusted. The skilled person will know how to perform such determinations. In an embodiment the determination of potassium is performed using the same hardware (magnet, pulse emitter, receiver and similar) as used in the sodium determination. Thereby the equipment and the set up can be economical feasible.

In an embodiment the method further comprises performing a vanadium determination of measurement on the petroleum fuel part using NMR, preferably by determining ⁵¹V isotope by performing at least one NMR measurement on the petroleum fuel part, obtaining at least one NMR spectrum from the NMR measurement(s) and performing at least one quantitative and/or qualitative vanadium determination. The NMR measurement preferably comprises obtaining at least one spin-lattice—T1 value and at least one spin-spin—T2 value of an exited vanadium isotope.

Vanadium determination using NMR can be performed in a similar manner as the method described above but by using other frequencies and optionally the strength of the magnetic field may also be adjusted. The skilled person will know how to perform such determinations. In an embodiment the determination of vanadium is performed using the same hardware (magnet, pulse emitter, receiver and similar) as used in the sodium determination. Thereby the equipment and the set up can be economical feasible.

In an embodiment the method comprises obtaining at least one NMR spectrum of ²³Na isotope and obtaining at least one NMR spectrum using an NMR equipment comprising an NMR spectrometer, comprising at least a magnet, a pulse emitter and a receiver, wherein the method further comprises obtaining at least one NMR spectrum of at least one other isotope using at least a part of the NMR equipment, the at least one other isotope is preferably selected from ³⁹K isotope and ⁵¹V isotope.

The method advantageously comprises performing NMR measurements of one or more isotopes on all of the petroleum fuel in an in-line process. Thereby a highly reliable determination can be obtained even where the petroleum fuel is highly inhomogeneous.

The petroleum fuel can I principle be any type of petroleum fuel. In general the method of the invention is in particular advantageous for use in performing quantitative determinations of sodium on inhomogeneous petroleum fuel such as heavy fuel oil (HFO), e.g. suitable for use as bunker fuel.

In an embodiment of the invention the method is combined with subjecting the petroleum fuel or a part thereof to a sodium removal treatment

In an embodiment the method comprises determining the concentration of sodium in the petroleum fuel, subjecting the petroleum fuel to a sodium removal treatment, and repeating the determination of concentration of sodium in the petroleum fuel.

The sodium removal treatment can be performed using any suitably method. The simplest sodium removal treatment method is to washing out sodium, e.g. by adding water, performing a through mixing of the petroleum fuel with the water and separating water, e.g. using centrifuging, sedimentation or other methods.

In an embodiment the method comprises
i. subjecting the petroleum fuel to a sodium removal treatment;
ii. determining the concentration of sodium in at least the part of the petroleum fuel;
iii. comparing the determined concentration of sodium with a selected limit, and
iv. if the determined concentration of sodium is larger than the selected limit, repeating steps i-iii, or if the determined concentration of sodium is below the selected limit, forwarding the petroleum fuel to combustion e.g. with intermediate storing in a day tank.

In step ii, the part of petroleum fuel subjected to sodium determination is advantageously withdrawn from the bottom of a storing tank. After determination and optionally sodium removal treatment the part of petroleum fuel is optionally returned to the storage tank and the method is continued until the sodium level is below a selected threshold.

The invention also relates to a system suitable for quantitative determination of sodium in petroleum fuel as described above.

The system of the invention comprises a NMR spectrometer, a digital memory storing a calibration map comprising calibrating data for calibrating NMR spectra obtained by the NMR spectrometer and a computer programmed to analyze the NMR spectra obtained by the NMR spectrometer using the calibration map and performing at least one quantitative sodium determination.

The spectrometer may be as described above and should preferably be configured to performing a NMR measurement of a petroleum fuel part of a suitable volume. The calibration map may be as described above.

The calibration map may be continuously updated with new data.

The system may comprise one, two or more computers, one, two or more spectrometers and/or one, two or more calibration maps.

The system may preferably be in data communication with the internet e.g. for communication with other similar systems, for sending and/or receiving data. The system may preferably comprise at least one display and/or an operating keyboard as well as any other digital equipment usually connected to digital systems, e.g. printers.

In an embodiment the system further comprises a digital memory storing a calibration map for one or more of the isotopes ³⁹K isotope and ⁵¹V isotope, the map comprises calibration data for said one or more isotopes and optionally of amounts thereof in petroleum fuels.

The system is advantageously configured to perform NMR measurement on a petroleum fuel part in flowing condition.

In an embodiment the system is configured to perform NMR measurement on a petroleum fuel part in form of a withdrawn sample.

In an embodiment the system is configured to perform a NMR measurement on a petroleum fuel part, and perform a quantitative sodium determination.

In an embodiment the system is configured to perform a NMR measurement on a petroleum fuel during fuelling e.g. to a vessel.

In an embodiment the system is configured to perform a NMR measurement on a petroleum fuel about to be injected into a gas turbine.

In an embodiment the system further comprises a sodium removal equipment, such as a sodium removal station, for removing sodium (purifying) from the petroleum fuel, the system is configured to perform a NMR measurement on a petroleum fuel about to be treated in the sodium removal equipment, and the system is further configured to perform a NMR measurement on a petroleum fuel after treatment in the sodium removal equipment (e.g. in form of a sodium removal station).

In an embodiment the system is configured to
i. subjecting the petroleum fuel to a sodium removal treatment;
ii. determining the concentration of sodium in at least the part of the petroleum fuel;
iii. comparing the determined concentration of sodium with a selected limit, and
iv. if the determined concentration of sodium is larger than the selected limit, repeating steps i-iii, or if the determined concentration of sodium is below the selected limit, forwarding the petroleum fuel to combustion e.g. with intermediate storing in a day tank.

The system may be configured to adjusting of one or more operating parameters of the sodium removal treatment based on the determination of the sodium removal treatment performance. Such adjusting may for example be an automated optimization.

The system may advantageously be arranged at a point of use such as near a gas turbine e.g. onboard a ship.

All features of the inventions including ranges and preferred ranges can be combined in various ways within the scope of the invention, unless there are specific reasons not to combine such features.

### BRIEF DESCRIPTION OF EXAMPLES AND DRAWINGS

The invention will be explained more fully below in connection with illustrative examples and embodiment and with reference to the drawings in which:

FIG. 1 is a schematic drawing of a system of the invention for determining sodium in a petroleum fuel in a fuel tank.

FIG. 2 is a schematic drawing of a system of the invention for determining sodium in a petroleum fuel under fuelling.

FIG. 3 is a schematic drawing of a system of the invention for determining sodium in a petroleum fuel transported from an petroleum fuel tank to a point of use.

FIG. 4 is a schematic drawing of a system of the invention for determining sodium in a petroleum fuel transported from one tank to another.

FIG. 5 is a schematic drawing of a system of the invention for determining sodium in a petroleum fuel withdrawn from a fuel tank.

FIG. 6 is a graph showing determination of concentration of sodium in bunker fuel, where all sodium was present in ionic form.

The figures are schematic and may be simplified for clarity. Throughout, the same reference numerals are used for identical or corresponding parts.

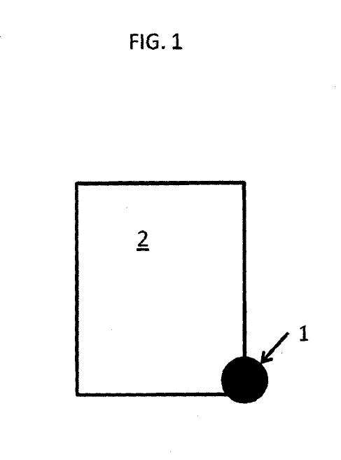

FIG. 1 is a schematic illustration of a system suitable for quantitative determination of sodium in a petroleum fuel, such as bunker fuel, using the method of the invention. The system comprises a NMR spectrometer 1, preferably as described above. The system further comprises a not shown digital memory storing a calibration map comprising calibrating data for calibrating NMR spectra obtained by the NMR spectrometer and a computer programmed to analyze the NMR spectra obtained by the NMR spectrometer using calibration map and performing at least one quantitative sodium determination.

The digital memory may be integrated in the computer. The spectrometer 1 is arranged to perform NMR measurements on the petroleum fuel in the fuel tank 2. Where the petroleum fuel is an inhomogeneous substance such a HFO e.g. a bunker fuel, it is normally desired to perform the sodium determination where it is expected to be higher, such a in corners and near the bottom. In the system shown in FIG. 1, the spectrometer 1 is specifically arranged to perform NMR measurement on a petroleum fuel part at the bottom and close to a corner of the fuel tank 2.

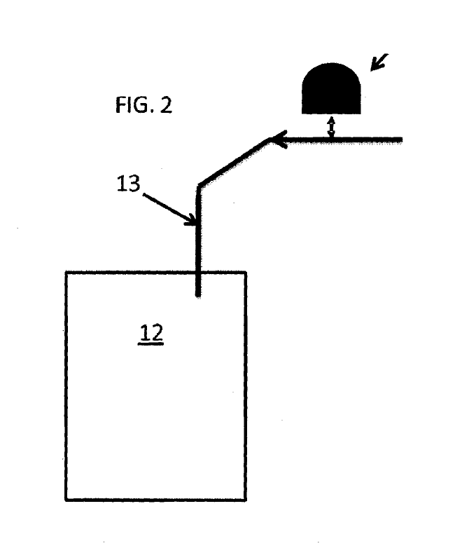

FIG. 2 is a schematic illustration of another system suitable for quantitative determination of sodium in a petroleum fuel according to the invention. The system comprises a NMR spectrometer 11, preferably as described above. The system further comprises a not shown a digital memory storing a calibration map and a not shown computer.

The spectrometer 11 is arranged to perform NMR measurements on the fuel in the pipe 13, which is under fuelling into fuel tank 12.

In a variation thereof the pipe section 13 comprises a not shown loop branched pipe section leading a part of the fuel to the NMR spectrometer 11 and back to the pipe section 13.

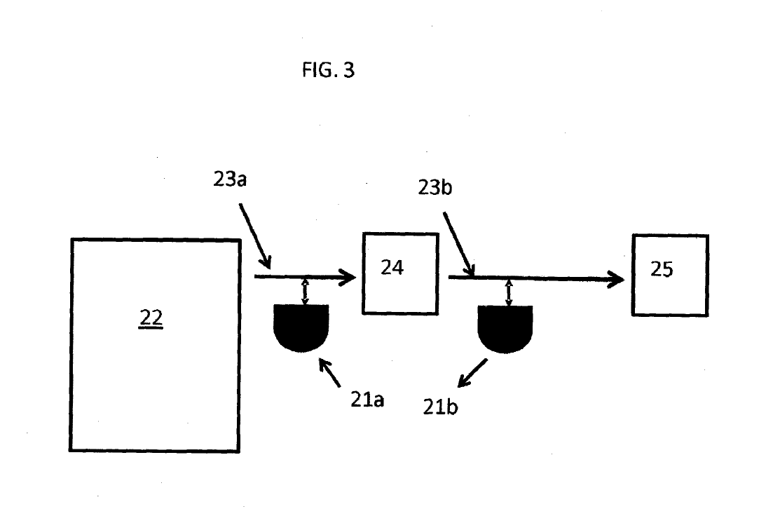

FIG. 3 is a schematic illustration of another system suitable for quantitative determination of sodium in a petroleum fuel according to the invention. The system comprises a first NMR spectrometer 21a, and second spectrometer 21b. The spectrometers are connected to a not shown digital memory and a not shown computer as described above.

The system further comprises a sodium removal station 24 for removing sodium, preferably as described above. The sodium removal station 24 advantageously comprises a mixing container, where the petroleum fuel is mixed thoroughly with water and a centrifuge, where the water—now with extracted sodium—is removed. The mixing container and the centrifuge may for simplification be an integrated unit. By using an integrated mixing container and centrifuge, the washing step can easily be repeated if required.

The system is configured to determinate the concentration of sodium in the fuel transported in the pipe 23a, 23b from a fuel tank 22 to for example an gas turbine via the pipe 23a and 23b. The fuel is transported via pipe section 23a where the first NMR spectrometer 21a is arranged to perform determinations of sodium.

The NMR spectrometer 21a may be arranged to perform determinations of sodium directly on the petroleum fuel flowing in the pipe section 23a. In a variation thereof the pipe section 23a comprises a not shown loop branched pipe section leading a part of the fuel to the NMR spectrometer 21a and back to the pipe section 23a.

The fuel is transported through the sodium removal station 24 for purification by removing at least some of the sodium. From the sodium removal station 24 the fuel is transported via pipe section 23b where the second NMR spectrometer 21b is arranged to perform determinations of sodium after the purification.

The NMR spectrometer 21b may be arranged to perform determinations of sodium directly on the fuel flowing in the pipe section 23ba. In a variation thereof the pipe section 23b comprises a not shown loop branched pipe section leading a part of the petroleum fuel to the NMR spectrometer 21b and back to the pipe section 23b.

From the second NMR spectrometer 21b the fuel is transported further e.g. to a point of use 25 e.g. a gas turbine.

The sodium determinations obtained from the first and the second NMR spectrometers 21a, 21b are compared and are used to determine the performance of the sodium removal station. The operating parameters of the sodium removal station 24, such as amount of water, temperature, mixing time and centrifugation condition can advantageously be adjusted based on the determinations obtained from the first and the second NMR spectrometers 21a, 21b.

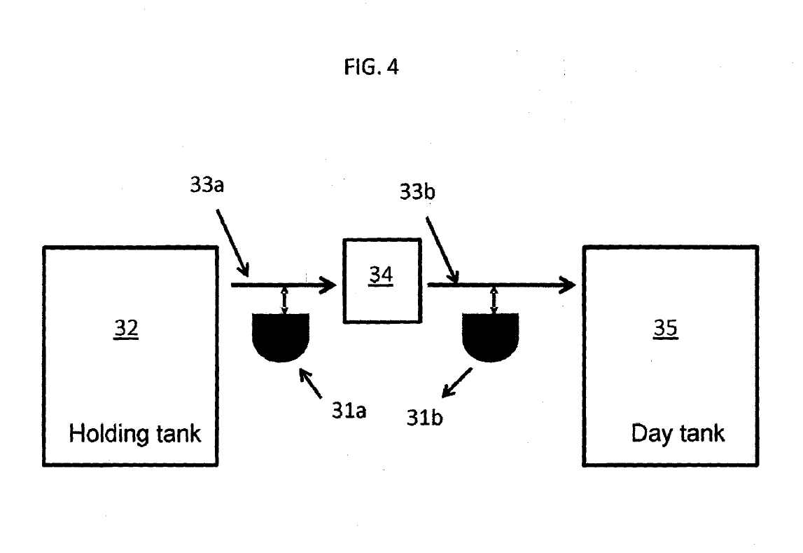

FIG. 4 is a schematic illustration of another system suitable for determination of the concentration of sodium according to the invention. The system comprises a first NMR spectrometer 31a, and second spectrometer 31b. The spectrometers are connected to not shown digital memory and computer as described above.

The system further comprises a sodium removal station 34 for removing sodium, preferably as described above.

The system is configured to perform quantitative determinations of sodium in a petroleum fuel transported in the pipes 33a and 33b from an fuel tank 32, e.g. a holding tank (storage tank) to a second fuel tank 32, e.g. a day tank.

A day tank is a fuel containment unit designed for installation in close-proximity to an engine, such as a gas turbine, to provide a reliable supply of fuel onboard a vessel. Although the holding tank 32 and the day tank 35 are drawn with same size in FIG. 4, it should be understood that a day tank is usually much smaller than a holding tank.

The fuel is withdrawn from the fuel tank 32 and is transported via pipe section 33a where the first NMR spectrometer 31a is arranged to perform determinations of sodium either directly on the pipe section 33a or on a not shown loop branched pipe section of pipe section 33a. The fuel will be transported through the sodium removal station 34 for washing out sodium. From the sodium removal station 34 the fuel is transported via pipe section 33b where the second NMR spectrometer 31b is arranged to perform determinations of sodium either directly on the pipe section 33b or on a not shown loop branched pipe section of pipe section 33b. From the second NMR spectrometer 31b the fuel is transported to the fuel tank 35, for example a day tank.

The sodium determinations obtained from the first and the second NMR spectrometers 31a, 31b are compared and are used to determine the performance of the sodium removal station as described above.

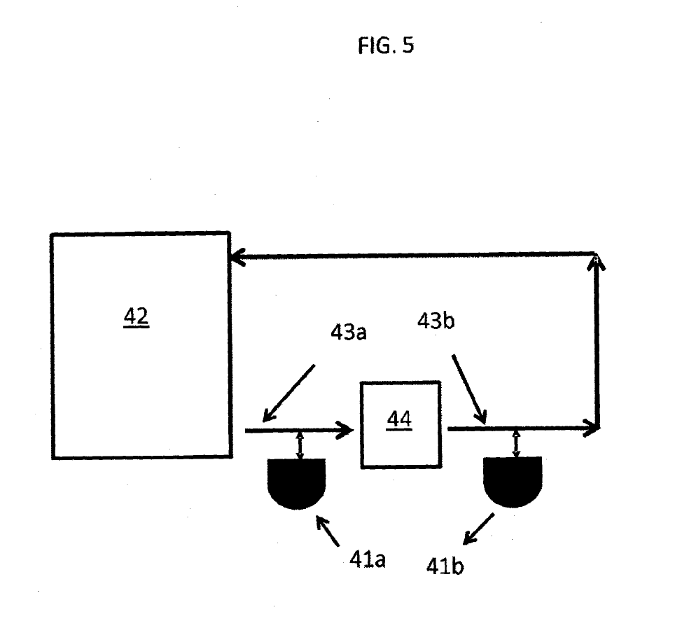

FIG. 5 is a schematic illustration of another system suitable for determining sodium in a petroleum fuel according to the invention. The system comprises a first NMR spectrometer 41a, and second spectrometer 41b. The spectrometers are connected to not shown digital memory and computer as described above.

The system further comprises a sodium removal station 44 for removing sodium, preferably as described above.

The system is configured to perform quantitative determination of sodium in the fuel transported in the pipe 43a and 43b from a fuel tank 42 to the same fuel tank 42.

The fuel is withdrawn from the fuel tank 42, preferably from a bottom part where the concentration is expected to be higher, and is transported via pipe section 43a where the first NMR spectrometer 41a is arranged to perform determinations of sodium either directly on the pipe section 43a or on a not shown loop branched pipe section of pipe section 43a. The fuel will be transported through the sodium removal station 44 for washing out sodium. From the sodium removal station 44 the fuel is transported via pipe section 43b where the second NMR spectrometer 41b is arranged to perform determinations of sodium either directly on the pipe section 43b or on a not shown loop branched pipe section of pipe section 43b. From the second NMR spectrometer 41b the fuel is transported back to the fuel tank 42, for example in a top part of the fuel tank 42.

### EXAMPLE 1

### Calibration Map

A statistically significant number of different heavy fuel oil samples with varying amounts of sodium are subjected to standard laboratory analysis of sodium using flame atomic absorption spectrometry. Each sample was separated into two equal portions A and B and the A samples were used for the flame atomic absorption spectrometry and the B samples were used for NMR analyses as described below.

The samples are selected such, that the spread of the concentration of sodium in theses samples cover the naturally found range, i.e. the range from 0 to 100 ppm or advantageously even higher, and preferably with several samples having concentrations within in the 0.5 to 2 ppm level. Also it is determined is the sodium is in ion form (dissolved) or if it is bound in compositions. Generally, most of the sodium in petroleum fuel will be in ion form, in particular where the petroleum fuel comprises significant amounts of water, which is usually the case with HFO.

The B samples are analyzed in parallel given the NMR based method described.

A correlation analysis of both datasets (laboratory (A) vs. NMR (B)) will show a correlation of the type y=a*x+b. The coefficients of this linear equation are used as a calibration map for calculating the true sodium content of a given sample from its NMR signal.

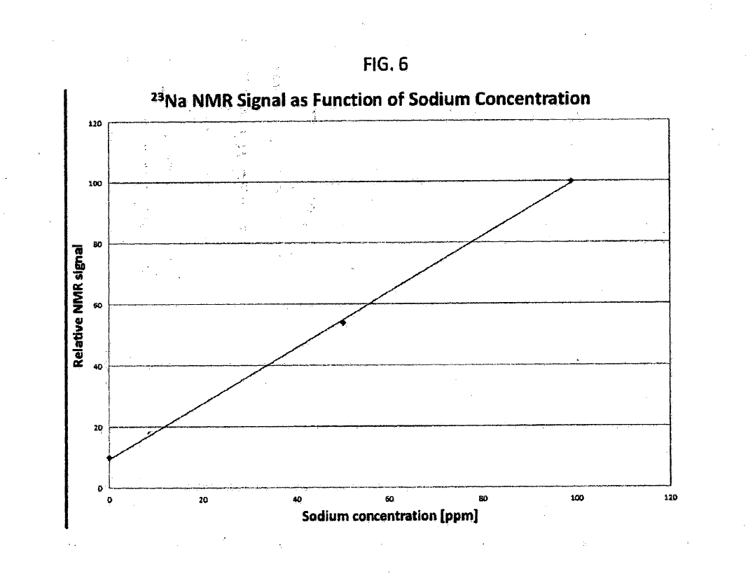

FIG. 6 shows the ²³NA NMR signal for sodium in ion form as a function of sodium concentration. It can be seen that the correlation is fully linear with the signal b as a constant background noise. Based on the graph of FIG. 6, NMR measurements performed on similar bunker fuel oils ant obtained under similar conditions can in a simple way be analyzed and the sodium concentration be determined.

### EXAMPLE 2

### NMR Measurement

A number of measurements were determined according to the method as shown below.

| | |
|---|---|
| Magnetic field strength | About 1.6 T |
| Petroleum fuel | Bunker fuel flowing in a pipe with an |
| | inner diameter of 12 mm and a |
| | velocity of about 1 l/min. |
| Measuring volume (petroleum fuel | 0.005 L |
| part) | |
| Exciting RF pulse | 90 degree pulse. |
| | Band width of about 200 KHz with |
| | centre about 16.5 MHz. |
| Refocusing pulses | Trains of 180 degree pulses. |
| | Band width of about 200 KHz with |
| | centre about 16.5 MHz. |
| TW | About 15 ms |
| TE | About 75 μs |
| Antenna Q (quality-factor) | About 50 |
| Exciting RF power | About 100 W |
| Measurement time | About 1 Hour |

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Some preferred embodiments have been shown in the foregoing, but it should be stressed that the invention is not limited to these, but may be embodied in other ways within the subject-matter defined in the following claims.

What is claimed is:

1-47. (canceled)

48. A method of quantitative determination of sodium in petroleum fuel, the method comprises determining a concentration of sodium in the petroleum fuel using NMR, the method preferably comprises determining sodium in the form of sodium isotope ²³Na by performing at least one NMR measurement on at least a part of the petroleum fuel, obtaining at least one NMR spectrum from the NMR measurement(s) and performing the quantitative determination of sodium based on the NMR spectrum.

49. The method of claim 48, wherein the NMR measurement is performed on the petroleum fuel in flowing condition, the NMR measurement preferably being performed on the petroleum fuel during transportation from a first container to a second container or to a point of use.

50. The method of claim 48, wherein the NMR measurement is performed in-line or semi-in-line, comprising performing the NMR measurement on-site.

51. The method of claim 48, wherein the petroleum fuel is or comprises a heavy fuel oil (HFO) suitable for use as bunker fuel.

52. The method of claim 48, wherein the NMR measurement comprises simultaneously subjecting the petroleum fuel to a magnetic field B, and a plurality of pulses of radio frequency energy E (RF pulses) and receiving electromagnetic signals from the ²³Na isotope.

53. The method of claim 52, wherein the magnetic field B is at least about 1 Tesla.

54. The method of claim 52, wherein the method comprises performing a plurality of NMR measurement at a selected magnetic field, the magnetic field is kept substantially stationary during the plurality of NMR measurements.

55. The method of claim 48, wherein the NMR measurement comprises simultaneously subjecting the petroleum fuel part to a magnetic field B, and an exciting RF pulse with frequencies selected to excite a nuclei spin of at least a part of the ²³Na isotope, wherein the frequency range of the exciting RF pulse comprises a band width of at least about 1 MHZ.

56. The method of claim 48, wherein the method comprises determining at least one relaxation rate of an exited ²³Na isotope.

57. The method of claim 56, wherein the method comprises determining at least one spin-lattice—T1 relaxation value of an exited ²³Na isotope.

58. The method of claim 56, wherein the method comprises determining at least one spin-spin—T2 relaxation value of an exited ²³Na isotope.

59. The method of claim 48, wherein the NMR measurement comprises simultaneously subjecting the petroleum fuel part to a magnetic field B, and a plurality of RF pulses wherein the RF pulses comprise
i. an exciting RF pulse, and
ii. at least one refocusing RF pulse.

60. The method of claim 59, wherein the exciting RF pulse is in form of a 90° pulse and wherein the refocusing RF pulse(s) is in the form of a 180° pulse.

61. The method of claim 59, wherein the refocusing RF pulse(s) is/are applied with an echo-delay time after the exciting RF pulse, the echo-delay time is of about 500 μs or less, more preferably about 150 μs or less.

62. The method of claim 59, wherein the at least one refocusing pulse comprises a plurality of refocusing pulses or trains of refocusing pulses applied with refocusing delay (TE) intervals between two consecutive refocusing pulses.

63. The method of claim 48, wherein the method comprises obtaining at least one NMR spectrum comprising an NMR spectrum from −500 ppm or less to +500 ppm or more.

64. The method of claim 48, wherein the method comprises determining quantitatively and/or qualitatively at least one compound comprising sodium and/or sodium ions.

65. The method of claim 48, wherein the method comprises determining the concentration of sodium ions.

66. The method of claim 48, comprising maintaining the temperature at a selected value or simultaneously determining the temperature.

67. The method of claim 48, wherein the determination of concentration of sodium in the petroleum fuel comprises providing a calibration map of petroleum fuels with known concentrations of sodium.

68. The method of claim 48, wherein the method comprises preparing calibration data, the calibration data is preferably stored on a digital memory, the method comprises feeding the NMR spectrum(s) or data obtained from the NMR spectrum(s) to a computer in digital communication with the digital memory and providing the computer to compare and analyze the data to perform at least one quantitative sodium determination.

69. The method of claim 48, wherein the method further comprises performing a potassium determination of measurement on the petroleum fuel part using NMR, preferably by determining ³⁹K isotope by performing at least one NMR measurement on the petroleum fuel part, obtaining at least one NMR spectrum from the NMR measurement(s) and performing at least one quantitative and/or qualitative potassium determination.

70. The method of claim 48, wherein the method further comprises performing a vanadium determination of measurement on the petroleum fuel part using NMR, preferably by determining ⁵¹V isotope by performing at least one an NMR measurement the petroleum fuel part, obtaining at least one NMR spectrum from the NMR measurement(s) and performing at least one quantitative and/or qualitative vanadium determination.

71. The method of claim 48, wherein the method comprises obtaining at least one NMR spectrum of ²³Na isotope and obtaining at least one NMR spectrum using an NMR equipment comprising an NMR spectrometer, comprising at least a magnet, a pulse emitter and a receiver, wherein the method further comprises obtaining at least one NMR spectrum of at least one other isotope using at least a part of the NMR equipment.

72. The method of claim 48, wherein the method comprises determining the concentration of sodium in the petroleum fuel, subjecting the petroleum fuel to a sodium removal treatment, and repeating the determination of concentration of sodium in the petroleum fuel.

73. The method of claim 72, wherein the method comprises
i. subjecting the petroleum fuel to a sodium removal treatment;
ii. determining the concentration of sodium in at least the part of the petroleum fuel;
iii. comparing the determined concentration of sodium with a selected limit, and
iv. if the determined concentration of sodium is larger than the selected limit, repeating steps i-iii, or if the determined concentration of sodium is below the selected limit, forwarding the petroleum fuel to combustion.

74. A system suitable for quantitative determination of sodium in petroleum fuel according to claim 48, the system comprises a NMR spectrometer, a digital memory storing a calibration map comprising calibrating data for calibrating NMR spectra obtained by the NMR spectrometer and a computer programmed to analyze the NMR spectra obtained by the NMR spectrometer using calibration map and performing at least one quantitative sodium determination.

75. The system of claim 74, further comprising a digital memory storing a calibration map for one or more of the isotopes ³⁹K isotope and ⁵¹V isotope, the map comprises calibration data for said one or more isotopes and optionally of amounts thereof in petroleum fuels.

76. The system of claim 74, wherein the system further comprises a sodium removal equipment for removing sodium from the petroleum fuel, the system is configured to perform a NMR measurement on a petroleum fuel about to be treated in the sodium removal equipment, and the system is further configured to perform a NMR measurement on a petroleum fuel after treatment in the sodium removal equipment.

77. The system of claim 76, wherein the system is configured to
i. subjecting the petroleum fuel to a sodium removal treatment;
ii. determining the concentration of sodium in at least the part of the petroleum fuel;
iii. comparing the determined concentration of sodium with a selected limit, and
iv. if the determined concentration of sodium is larger than the selected limit, repeating steps i-iii, or if the determined concentration of sodium is below the selected limit, forwarding the petroleum fuel to combustion.
